(19) United States
(12) Patent Application Publication
(10) Pub. No.: US 2025/0268519 A1
(43) Pub. Date: Aug. 28, 2025

(54) DEVICE AND METHOD FOR MEASUREMENT OF PHYSIOLOGICAL PARAMETERS BY TEMPORARY CONTACT WITH A RECEPTOR SURFACE

(71) Applicant: NANOMADE LAB, TOULOUSE (FR)

(72) Inventor: Adrien THIRION, TOULOUSE (FR)

(73) Assignee: NANOMADE LAB, TOULOUSE (FR)

(21) Appl. No.: 18/692,930

(22) PCT Filed: May 26, 2022
(86) PCT No.: PCT/EP2022/064356

§ 371 Date: Aug. 13, 2024

(30) Foreign Application Priority Data
May 26, 2021 (FR) FR2105480
Oct. 21, 2021 (FR) FR2111154

(51) Int. Cl. A61B 5/00; A61B 5/0205; A61B 5/08; A61B 5/11

(52) U.S. Cl. CPC A61B 5/4561; A61B 5/0205; A61B 5/0816; A61B 5/1102; A61B 5/6891; A61B 5/6892; A61B 2562/0247; A61B 2562/0252; A61B 2562/046

(57) ABSTRACT

The invention relates to a device and a method for evaluation of the physiological parameters of an individual in brief contact with said device, comprising: —a pressure sensor (121, 122) comprising a flexible test body (230) and elementary gauges (220) sensitive to the deformation of said test body; —a receptor surface (111, 112) able to come into contact with the individual and to transmit the forces resulting from this contact to the pressure sensor (121, 122); —computing means able to process the signals (520) coming from the pressure sensor, and a program implementing said computing means in such a way as to determine at least one item of information from among: —the breathing rate of the individual; —the heart rate of the individual; —the posture of the individual; —their development over time, and to generate an alert depending on these items of information.

### TECHNICAL FIELD

This document contains the description of the objects and methods described in provisional patent application FR2105480 filed on 26 May 2021 for which it claims priority, with the addition of improvements.

The invention pertains to a method and a device for measuring physiological parameters of an individual by a temporary contact with a receptor surface. The term temporary, characterizes a non-portable device, unlike so-called wearable devices of the prior art such as wrist bands, chest belts, glasses, belt boxes, helmets etc.

### BACKGROUND ART

The measurement of physiological parameters such as heart rate, respiratory rate, blood pressure and their temporal changes, makes it possible to detect, even to anticipate and prevent, risky situations for an individual, ranging from relatively benign risks such as stress to risk factors such as cardiovascular accidents.

In particular, the COVID-19 pandemic leads to the isolation of people as part of containment measures aimed at reducing the circulation of the virus, particularly with regard to the elderly.

The constraints resulting from these measures, and in particular associated with the phenomenon of global warming and the increase in the frequency of summer heat waves, endanger isolated people and more particularly elderly people with cardiovascular, pulmonary or metabolic problems, who have even more difficult access to air-conditioned places.

Remote assistance provides the beginning of a solution to these situations. Thus, the measurement of physiological parameters such as body temperature, heart rate, blood pressure and respiratory rate of the person of concern is associated with the measurement of environmental parameters such as temperature and humidity and may be transmitted to a remote monitoring center in which operators, associated or not with an artificial intelligence analysis system, assess the situation.

If a potentially risky situation is detected, an intervention protocol is triggered, including, for example, a phone call to the person in order to ensure that everything is fine and advise her, if necessary, on a course of action.

The document Alex Buoite Stella et al. “Heat risk exacerbation potential for neurology patients during the COVID-19 pandemic and related isolation” International Journal of Biometrology (2021) 65:627-630-Nov. 8, 2020, describes such a remote assistance device.

The difficulty of these remote assistance devices lays in the collection of physiological information.

Devices, frequently referred to as “wearables”, such as a wrist band or a chest belt, can measure such parameters relating to the physical-psychic state of the person wearing them.

In a medical environment, different types of sensors can be installed on a patient to monitor physiological parameters via an electrocardiogram (ECG), an electroencephalogram (EEG), a respiratory or blood pressure monitoring.

However, these solutions are not suitable for regular and long-term collection, due to the discomfort they produce in daily activities and the feeling of surveillance they raise by their simple presence.

Indeed, most parameters of interest require for their measurement sensors in contact with the skin of the individual and are themselves responsive to environmental parameters such as temperature or humidity.

Document U.S. Pat. No. 10,292,658 describes a device integrated into a toilet seat and comprising sensors coming into contact with a user's skin in order to measure physiological parameters, including an electrocardiogram (ECG).

In addition of having to be in contact with the skin, such a device for the acquisition of a person's electrocardiogram, as well as the parameters that can be derived from it, is very responsive to humidity both ambient and at the level of contact with the electrodes, especially in the presence of sweat, so that this technique, which is reliable in the laboratory, is delicate to implement in a stand-alone sensor, and more particularly if the latter is not a wearable.

### SUMMARY OF THE INVENTION

The invention aims at solving the shortcomings of the prior art and to this end pertains to a device for assessing the physiological parameters of an individual in temporary contact with the device, which device comprises:
a pressure sensor comprising a deformable test body and a plurality of elemental gauges of high sensitivity linked to the test body and responsive to the deformation of the test body;
a receiving surface capable of coming into contact with the individual and transmitting a force resulting from this contact to the pressure sensor;
computer means, capable of processing a signal coming from the pressure sensor and a computer program for determining at least one item of information among:
a respiratory rate of the individual and an evolution of this respiratory rate;
a heart rate of the individual and an evolution of this heart rate;
a posture of the individual and an evolution of this posture;

and generating an alert according to the information.

A temporary contact means a contact time of less than 120 seconds, being it understood that the device of the invention is also capable of evaluating the parameters referred to over much longer durations.

An elemental gauge of high sensitivity means a strain gauge using a technology such as to obtain a gauge factor greater than 10 over the deformation measurement range that is required for the implementation of the device.

The gauge factor is the ratio between the variation of an electrical property measured at the terminals of the gauge, generally a resistance, and the variation of deformation of this gauge.

The invention may be implemented according to the embodiments and variants exposed hereafter which are to be considered individually or according to any technically operative combination.

According to an advantageous embodiment, a gauge of the plurality of elemental gauges comprises a conductive nanoparticles assembly in an insulating ligand, grafted on a substrate, the substrate being bonded to the test body.

The use of this gauge technology makes it possible to achieve a gauge factor of the order of 80 over a deformation range of +1% and thus to ensure the detection of a heartbeat or a breathing of the individual.

According to an advantageous embodiment, the test body comprises a polycarbonate plate with a thickness less than or equal to 0.5 mm comprising strips delimiting cutouts, the elemental gauges being positioned at the intersections of these strips.

This type of test body is easy to integrate into the backrest or seat of a chair or into a mattress without impairing the comfort of the item in which it is integrated.

According to a preferred embodiment, the pressure sensor comprises 12 elemental gauges.

According to an embodiment, the device is a chair and the sensor comprises a backrest sensor inserted into a backrest of the chair, and the receiving surface comprises a backrest receiving surface.

Advantageously, the backrest of the chair comprises a padding.

Advantageously, the sensor comprises a seat sensor inserted into a seat of the chair, and the receiving surface comprises a seat receiving surface.

Advantageously, the seat comprises a padding.

The embodiment of the device into a chair is more particularly, but not exclusively, suitable for monitoring the physiological parameters of an individual driving a transportation or a work craft.

According to another embodiment, the device is a mattress, the pressure sensor comprises a sensor inserted into the mattress and the receiving surface is a sleeping surface of the mattress.

This embodiment is more particularly adapted for the implementation of a home hospitalization with telemedicine.

The invention also relates to a method for measuring at least one physiological parameter of an individual, implementing the device of the invention, comprising a step of acquiring and digitizing a signal from the plurality of elemental gauges, comprising a continuous part and a pseudo-periodic part, the method comprising over a processing time window, at least one of the steps of:
measuring a pressure exerted by the individual on the gauges of the plurality from the continuous part of the signal after its adjustment for its temporal drift on a processing time window and deducing therefrom a posture of the individual;
during a pre-processing step, extracting the pseudo-periodic part of the signal for removing an effect of movements of the individual, carrying out a smoothing of the pseudo-periodic signal in a smoothed signal and assessing a respiratory frequency of the individual by a time distance between two peaks of the smoothed signal;
filtering by a bandpass filter with cut-off frequencies of 0.5 Hz and 20 Hz the pseudo-periodic part of the signal obtained by the pre-processing step, selecting a signal thus filtered coming from a gauge of the plurality having a better signal-to-noise ratio, exploring the selected signal by a sliding window and detecting in the sliding windows 2 patterns corresponding to heart beats and the time distance between these patterns, and assessing a heart rate from these data.

This method makes it possible to assess 3 physiological parameters of interest from the same device.

### BRIEF DESCRIPTION OF THE DRAWINGS

The invention is implemented according to the preferred embodiments, in no way limiting, exposed hereafter with reference to FIGS. 1 to 11 in which:

FIG. 1 shows in a perspective view an exemplary embodiment of the device of the invention in a chair;

FIG. 2 shows, in an exploded perspective view an exemplary embodiment of a pressure sensor implemented in the device of the invention;

FIG. 3 schematically represents, according to an exploded perspective view, an exemplary embodiment of an elemental gauge of a sensor of the device of the invention;

FIG. 4 is a flowchart of the method of the invention.

FIG. 5A shows an example of a digitized raw signal received from all the elemental gauges of a sensor of the device of the invention;

FIG. 5B shows an example of the digitized raw signal on an elemental gauge and illustrates the processing performed on this signal;

FIG. 6 shows an example of a signal processed for the determination of a respiratory rate;

FIG. 7 illustrates a processing performed on the signal to obtain a BCG, in order to eliminate the influence of micromovements;

FIG. 8 shows examples of signal processing to select the elemental gauge signal that conveys the most information for a BCG analysis;

FIG. 9 Illustrates the processing performed in a BCG signal exploration window in order to determine the distances between peaks;

FIG. 10 illustrates a signal processing from an elemental gauge for the analysis of the posture of the individual in contact with the device of the invention;

FIG. 11 shows an exemplary implementation of the device of the invention to a mattress.

### DESCRIPTION OF EMBODIMENTS

The invention consists of the combination of a pressure sensor with a set of specific characteristics, with a suitable signal processing method.

Indeed, the pressure sensor used by the invention exhibits a combination of:
high sensitivity;
extended deformation measurement range,
mechanical flexibility allowing it to be integrated, without discomfort for the user, into any piece of furniture comprising a receiving surface, with or without padding.

These characteristics enable the sensor to detect the presence of an individual in contact with the receiving surface of a piece of furniture thus equipped, even to detect the posture of this individual by the distribution of pressure on the receiving surface, but also to measure the pressure variations generated on this receiving surface by the heartbeat, blood circulation and breathing of this individual regardless of the weight of the latter.

Thus, according to non-limiting embodiment examples, the receiving surface is:
a backrest or a seat of a chair or a bench with or without padding;
a sleeping surface of a bed or a berth;
an area delimited on a floor, covered or not with a carpet or a coating.

Consequently, the device of the invention is applicable in any furniture, at home or onboard, in particular in a driver or passenger seat in the field of transportation, work equipment, sport and leisure or devices intended for people with reduced mobility, in a mattress, in particular intended for a hospital bed or for a home hospitalization.

The sensitivity of the pressure sensor implemented by the invention and the associated method make it possible to establish a balistocardiogram (BCG) of the person coming into contact with a receiving surface thus functionalized.

Compared to an ECG or electrocardiogram, BCG is much less responsive to environmental parameters such as humidity and does not require direct contact with the individual's skin.

On the other hand, the measurable signal is more responsive to

phenomena such as noises, vibrations or changes in the posture of the person in contact with the receiving surface, and in general, the signal-to-noise ratio of the relevant information is less favorable than for an ECG. This drawback is solved by the method of the invention.

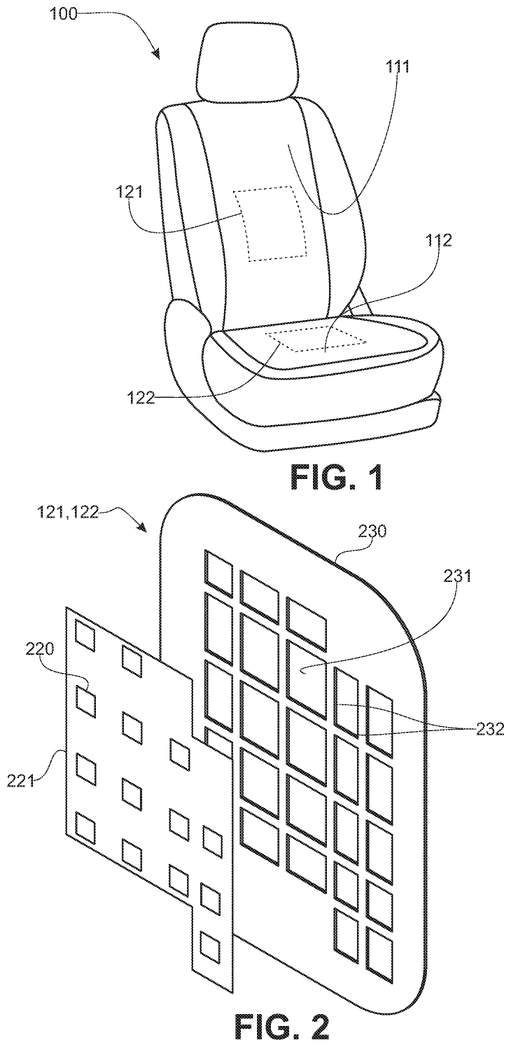

FIG. 1, according to an exemplary embodiment, a piece of furniture such as a chair (100) configured for implementing the device and the method of the invention, comprises one or more receiving surfaces (111, 112) aimed to coming into contact with a body part of a user when the latter uses the piece furniture.

According to this exemplary embodiment, the backrest and seat of the chair are receiving surfaces with padding, into which pressure sensors (121, 122) are inserted.

FIG. 2, according to an exemplary embodiment, the pressure sensor (121, 122) of the device of the invention, comprises one or more elemental strain gauges (220) attached, for example by bonding, to the back of a thin polycarbonate plate (230), for example with a thickness of less than or equal to 0.5 mm, acting as a test body. The elemental gauges (220) are deposited on a thin insulating substrate (221) for example using a capillary/convective deposition technique or by soft lithography.

According to an exemplary embodiment, the elemental gauges (220) are arranged on the back of the polycarbonate plate (230) so as to be protected by the polycarbonate, for example at the intersections of the strips (232) delimiting the cuts (231).

According to a particular example, the sensor (121, 122) comprises 12 elemental gauges.

The sensor further comprises a circuit and electrical connections (not shown) suitable for acquiring the information delivered by the elemental gauge(s) (220), the circuit and the connections being, at least in part, deposited on the substrate (221), also by soft lithography or photolithography techniques, according to exemplary implementations.

The polycarbonate plate (230) thus equipped is, for example, integrated into the padding of a seat or a backrest of a chair, the face opposite the one comprising the elemental gauge(s) (220) being turned towards the individual likely to use the chair.

The low thickness of the sensor and its shape, including for example cut-outs (231) improving its flexibility, does not cause any discomfort and does not impair the comfort of use of that chair compared to an ordinary chair.

According to another embodiment (not shown), corresponding to the case where the piece of furniture comprises a rigid receiving surface, for example the backrest of a chair, the elemental gauges are directly attached to the rigid surface, for example on the back of the backrest, protected from the environment by a protective coating.

The extreme sensitivity conferred to the elemental gauges by the exploited physical phenomenon allows this type of configuration.

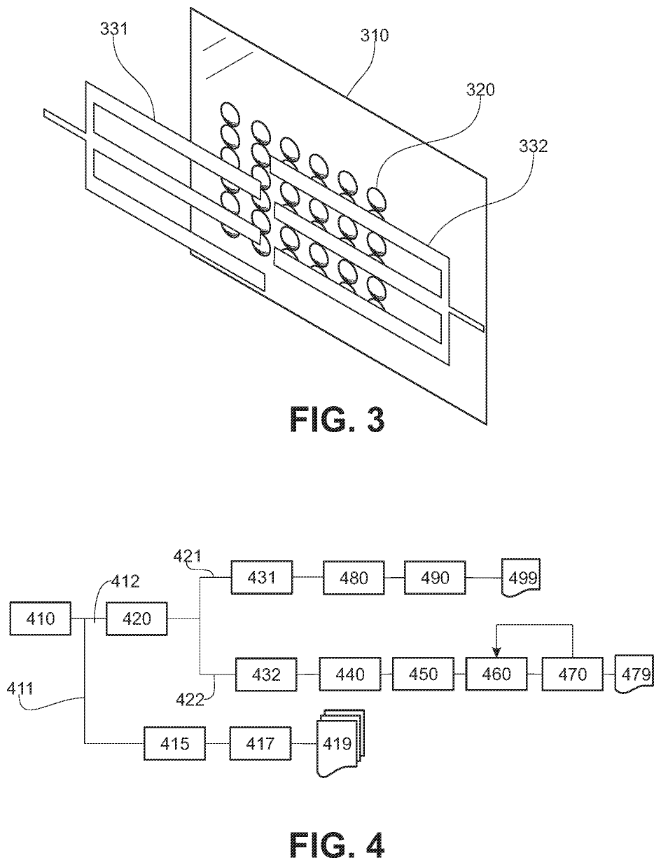

FIG. 3, according to an exemplary embodiment, an elemental gauge (220) comprises a substrate (310) on which is deposited an assembly of electrically conductive or semiconductive nanoparticles (320) in an electrically insulating ligand capable of binding to the surface of the nanoparticles.

By way of non-limiting examples, the nanoparticles (320) consist of zinc oxide (ZnO) or tin-doped indium oxide (In₂O₃—SnO₂), or ITO. For example, the substrate (310) consists of ethylene poly (terephthalate) (PET), the ligand is for example based on phosphonic acid.

The nanoparticles are attached to the substrate by a graft, by means of a chemical coupler, for example a silane.

These deposition techniques, both assemblies of nanoparticles and electrodes or electrical circuit elements, are known from the prior art, in particular from documents U.S. Pat. Nos. 9,436,215 B2 and 10,318,143 B2 and are not described further.

Two conductive electrodes (331, 332), for example made of ITO, in the form of combs are deposited on the assembly of nanoparticles (320) in a nested configuration, called interdigitated, that is to say that the teeth of one of the comb electrodes are interposed between the teeth of the other comb electrode.

Thus, according to this exemplary embodiment, each tooth of a comb juxtaposed with a tooth of the other comb defines between the teeth a micro strain gauge which is the place of electrical conduction by tunneling between the nanoparticles of the assembly located between the electrodes delimiting the micro-gauge. The conduction varies according to the distance between the nanoparticles of the assembly, which distance is a function of the pressure applied to the nanoparticles assembly or more generally to the deformation to which the gauge (220) is subjected.

Thus, the conductivity or resistance of the gauge (220) varies with this deformation.

A passivation layer (not shown) consisting, for example, of a polyimide, is placed on this stack in order to protect it from the environment, in particular from moisture.

The gauge factor defines the ratio of the relative change in the resistance of elemental gauge ΔR/R0 according to the relative deformation of the gauge. This gauge factor easily reaches 80 or more over a deformation range of +1%, the resistance R0 of such a gauge comprising nanoparticles of ITO in a ligand based on phosphonic acid, exceeds 2000 ohms in the absence of deformation.

Such an elemental gauge (220) exhibits a high sensitivity and the sensitivity of the sensor is further improved by the combination of several of these elemental gauges with a suitable test body.

This sensitivity makes it possible to detect the presence of an individual on the piece of furniture, the movements or micromovements of this individual, his posture and his changes in posture, his heart rate by BCG and the variation of this heart rate, as well as the respiratory rate of this individual and its variation.

The person skilled in the art understands that this various information is contained in a signal originating from the sensor and that it shall be extracted from this signal by an adapted processing which is also part of the invention.

This processing is carried out by computer means including, according to a known general configuration, means of acquisition and digitization of signals, means of calculation and means of memory, all being controlled by a computer program.

Such IT means include a clock so that any acquisition and storage of data, raw or processed, can be associated with a date and that this date can be used for any processing, in particular those aimed at determining an evolution.

FIG. 4, according to a first digitizing step (410) of the method of the invention, the signals from each elemental gauge of the sensor are digitized according to methods known to the prior art in order to later subject them to adequate digital processing.

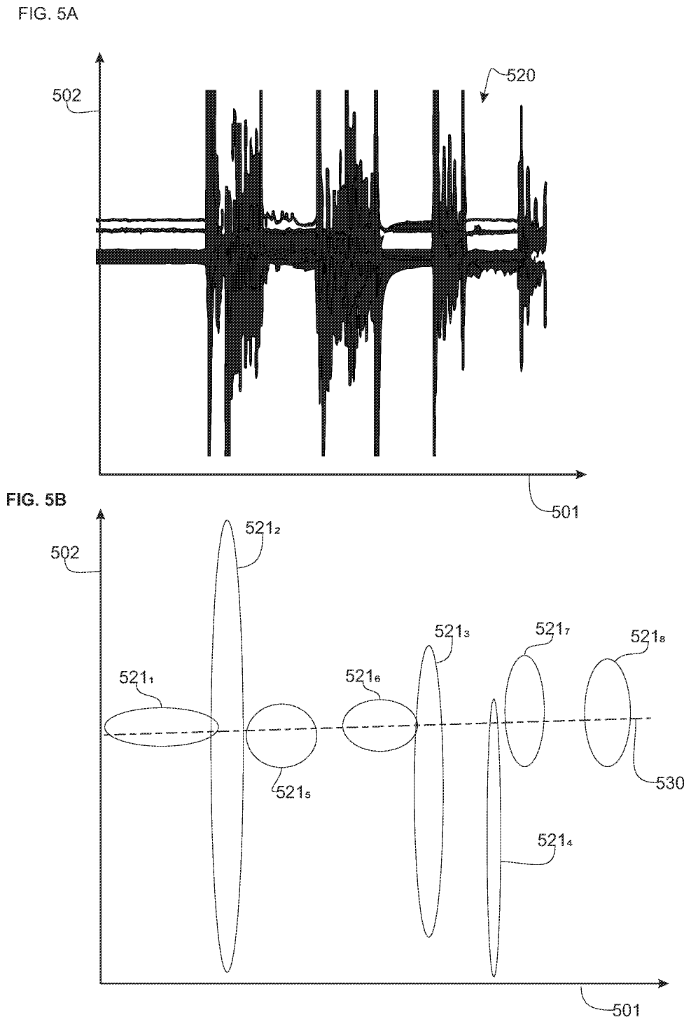

FIG. 5A shows an example of the change in the value (502) of raw signals (520) as a function of time (501), these raw signals (520) may represent pressures or accelerations.

Thus, in a graph showing a sensor emitted signal (520) amplitude (502), for example in volts, as a function of time (501), wherein each elemental gauge emits such a signal comprising a combination of different aimed information (movements, posture, heart rate, respiratory rate), each of this information produces events that differ in amplitude, frequency, and reproducibility.

Thus, for example, heartbeats as well as breathing correspond to pseudo-periodic events, while the movements or micromovements of the individual are more stochastic, the movements producing variations of higher amplitude and micromovements producing information whose amplitude is between that of the heartbeats and that of breathing.

For example, FIG. 5B, the observation of the raw signal (521) corresponding to a single elemental gauge makes it possible, under experimental conditions, to identify specific events. A first portion (521₁) of this signal corresponds to the absence of contact of the individual with the receiving surface.

The next portion (521₂) corresponds to the detection of a movement, for example, when the individual sits in the chair of FIG. 1.

Other events (521₃, 521₄) also correspond to movements of the individual.

The pseudo-periodic parts (521₅, 521₆, 521₇, 521₈) of the signals correspond to breathing signals, while the signal portions between these breathing phases correspond to a situation where the subject is in contact with the receiving surface but blocks his breathing.

The signals corresponding to the heart rate and possible micromovements are present but are not visible graphically at the scale of FIG. 5B.

The time drift of the signal of the corresponding gauge is estimated by a linear regression producing a line (530) over a defined time window, typically greater than 3 seconds and less than 60 seconds and in the order of 20 seconds.

FIG. 10, similar to FIG. 5B, corresponds to an exemplary observation of the amplitude (1002) of a signal (1020) corresponding to an elemental gauge as a function of time (1001), from which the time drift has been deduced.

By comparing the average signal level (1021₁) before the individual comes into contact with the piece of furniture to the average signal level (1021₂) after the individual comes into contact with the piece of furniture, the difference (1030₁) gives the corresponding pressure exerted on the elemental gauge from which the signal is originated.

Then, during a subsequent movement of the individual, to see if this pressure (1030₂) changes.

By performing this operation on all the elemental gauges of the sensor, and by successive time windows, the distribution of these measurements provides information on the posture of the individual, that is to say on the distribution of the pressure that he exerts on the piece of furniture (chair, bed . . . ) and consequently, the evolution of this information over time, provides information on the changes, or stays, of the individual posture.

According to non-limiting examples, this analysis is useful for detecting a risk of bedsores in the case of the follow-up of a bedridden person, for example in home care. Those bedsores are a result of a relatively constant pressure at the same points over a long period of time. In another example, this posture analysis is useful for detecting drowsiness or fainting of the individual when driving a vehicle or a craft.

Returning to FIG. 4, the signal may be processed according to two branches.

According to an optional processing branch (411) aimed at extracting information relating to the posture of the individual on the piece of furniture, during a resetting step (415) the drift of the signal is estimated over a defined time range, typically in the 20 seconds order of magnitudes, for example by a linear regression over the entire raw signal, and the corresponding straight line is deduced from the signal over the duration of the defined time range.

During an assessment step (417), the average pressure, the average level differences of this corrected signal in successive observation time windows are compared, these differences are stored in a timestamped file (419) for each elemental gauge.

The data in this file (419) may then be analyzed according to a temporality specific to the application of the device, to derive information on the individual's posture and its evolution, and lead to the generation of alerts according to the result of this analysis.

According to another branch (412) the processing aims at extracting from the signal information relating to a heart rate and to a respiratory rate, this branch (412) comprises a step (420) of pre-processing raw signals issued by the elemental gauges of the sensor.

The pre-processing carried out during this step (420) aims to separate the influence on the signals of the part corresponding to pseudo-periodic phenomena such as breathing and heart rate, from the part related to movements of the individual in contact with the receiving surface. According to this example, this step may be carried out by an analysis of a signal amplitude threshold.

Thus, to isolate the pseudo-periodic part of the signals, the signals of all the elemental gauges are set to 0 each time a peak in the digitized raw signal crosses a defined threshold and for a duration around this peak, for example for a duration ranging from 1 second before the phenomenon (peak) to 3 seconds after this phenomenon.

For example, a peak in the signal is assigned to a movement if the amplitude of the signal (max-min) over a range of 1 second exceeds a certain threshold for signals from at least two elemental gauges and preferentially from at least 3 elemental gauges.

After this pre-processing step (420) the majority of the peaks attributable to movements are eliminated from the signal. The person skilled in the art understands that the signal corresponding to these movements can be isolated from the raw signal by proceeding in a similar way.

This pre-processing method makes it possible to eliminate signals that are irrelevant to the intended objective, in this case signals corresponding to movements, when the intended analysis is focused on BCG or respiratory rate, without shifting the phase of the signal according to the frequency.

Starting from this pre-processing step, the signal may follow two processing branches (421, 422) one branch (421) corresponding to a processing aiming at a respiratory rate assessment and another branch (422) for a processing aiming at a BCG assessment.

Thus, on the branch (421) corresponding to the processing of a respiratory rate, the pre-processed signal is the subject of a smoothing step (431), for example via a Savitzky-Golay algorithm with a 3^{rd} degree polynomial and a smoothing window of 1 second.

On the branch (422) corresponding to the processing of a BCG, the pre-processed signal is the subject of a filtering step (432) in the form of a band pass filter with exemplary cut-off frequencies of 0.5 Hz and 20 Hz.

According to exemplary embodiments, this filtering step (432) on the BCG processing branch (422) implements a Butterworth or Savitzky-Golay type filtering without these examples being limiting.

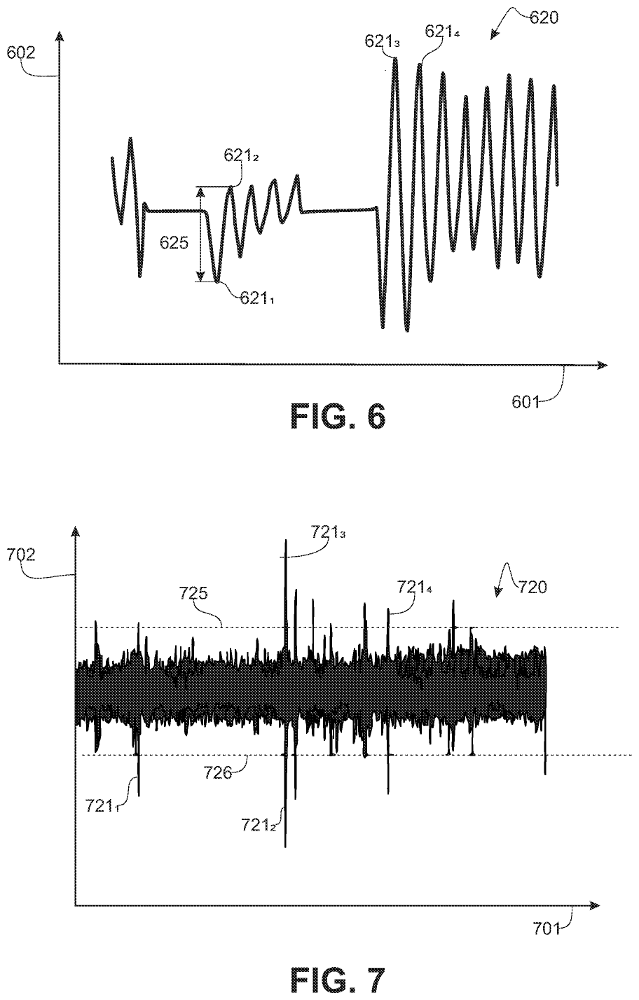

FIG. 6, according to an example, the signal (620) pre-processed according to the smoothing step (431) comprises peaks (621₁, 621₂, 621₃, 621₄) appearing as a function of time (601) in the signal amplitude (602), corresponding to respiratory events (inhaling or exhaling).

For example, a respiratory event corresponds to a succession of two successive peaks (621₁, 622₂), oriented in opposite ways, and separated by a minimum amplitude (625), defined by experience.

As a non-limiting example, the minimum amplitude (625) for the selection of significant peaks is defined relative to the signal.

Thus, the minimum amplitude (625) for peak selection is equal to ⅕^{th} of the maximum amplitude of the signal over a given measurement range.

The duration of this measurement range is chosen inside a time frame of 2T_max, defined below, i.e. of the order of 3 seconds and a maximum of 60 seconds, preferably around 20 seconds.

Thus, going back to FIG. 4, a step (480) of detecting the breathing peaks, for example using the method described above, follows the smoothing step (431) of the signal.

According to a step (490) of estimating the individual's respiratory rate, the respiratory rate is determined from the results of the peak detection step (480) on successive time windows and the corresponding results are recorded in a timestamped file (499).

Thus, by way of example, FIG. 6, to determine a respiratory rate, the time distances between two successive valid peaks (621₂, 621₃, 621₄) are determined over a defined time range, the respiratory rate is determined by taking the inverse of the median of these distances over the given time range.

FIG. 4, the values relating to the individual's respiratory rate, stored in the file (499) can be compared according to a periodicity specific to the application, to acceptable values depending on the age of the individual using the device of the invention and may lead to generating alerts based on these results.

A branch (422) of the method corresponds to the processing of a BCG.

The mechanical phenomena corresponding to a BCG and captured by the elemental gauges produce a lower signal-to-noise ratio than those relating to breathing and therefore require more sophisticated processing in order to isolate cardiac phenomena and assessing the heart rate and its evolution.

Movements were detected during the pretreatment step (420). The signal thus pre-processed is analyzed to determine the BCG only if:
it is free of movement phenomena for a predefined duration comprised between 2T_max (3 seconds) and 60 seconds, preferably 20 seconds, and
the presence of the individual on the receiving surface is confirmed.

As the movements are eliminated and the signal filtered, the signal is still likely to include information corresponding to the micromovements of the individual.

Thus, during a micromovement identification step (440), the pre-processed signal is filtered and analyzed.

FIG. 7, in an exemplary time (701)—amplitude (702) diagram, showing the pre-processed and filtered signals (720) from the elemental gauges, the peaks (721₁, 721₂, 721₃, 721₄) exceeding a given threshold (725, 726) are attributed to micromovement of the individual in contact with the receiving surface of the device.

The thresholds (725, 726) are defined by tests and, according to embodiment variants, are functions of the age, weight and state of health of the individual, either from tables established according to these criteria or by calibration tests carried out with the cooperation of the concerned individual.

According to alternative embodiments, the thresholds also take into account the environment of the device such as the presence of vibrations, in particular when the device of the invention is intended to be installed in a transportation mean.

Thus, to isolate the pseudo-periodic part of the signals, the signals of all the elemental gauges are set to 0 each time a peak (721₁, 721₂, 721₃, 721₄) in the signal (720) crosses a defined threshold and for the duration of this peak, as well as for a defined time range around such a peak.

Through this processing, the influence of micromovements is eliminated from the signal intended for a BCG analysis.

The BCG is responsive to the posture of the person on the receiving surface so that, according to this posture, some elemental gauges of the sensor are more responsive than others to the heartbeat of the individual.

To this end, during a selection step (450), a sensor or sensors with the best signal are selected.

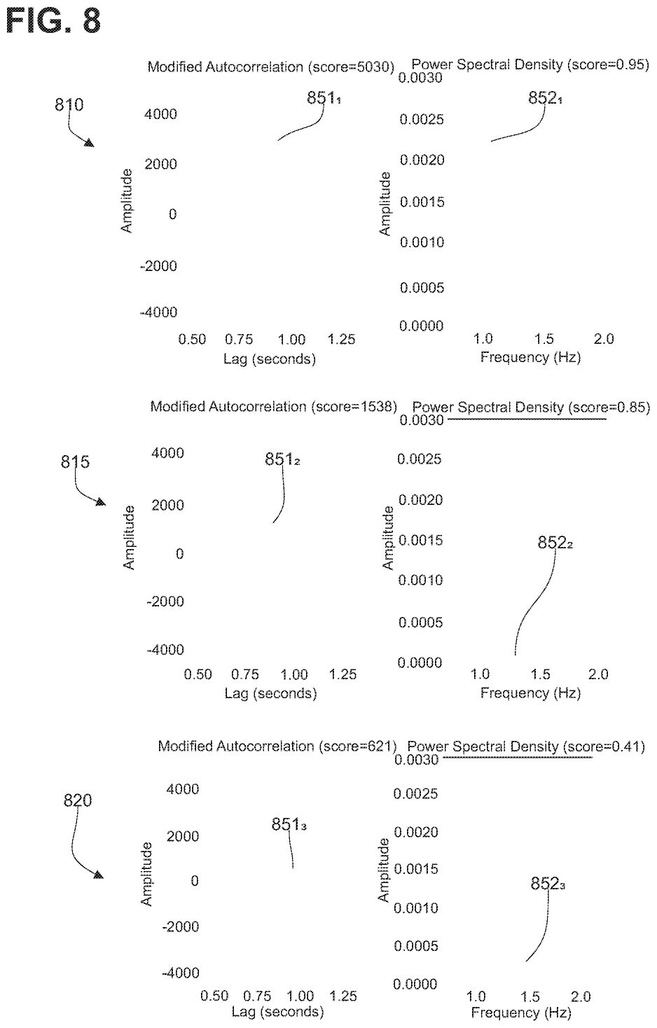

FIG. 8, for this purpose an autocorrelation function (851₁, 851₂, 851₃) is calculated on the signals from each elemental gauge of the sensor of the device of the invention, over a defined time range, namely between 3seconds (2T_max) and 60 seconds and preferably around 20 seconds according to an exemplary embodiment.

As a reminder, the signals, following the previous treatments, are free of phenomena related to movements or detectable micromovements, and do correspond to signals emitted when the individual is in contact with the receiving surface.

Additionally, a spectral analysis (852₁, 852₂, 852₃) of the signal is performed.

The signals of the elemental gauges are classified according to the amplitude of the autocorrelated signal and the 2 majority peaks of the spectrum in a frequency range. If there are not 2 sufficiently large peaks, the criterion is calculated by the integral of the spectrum in a frequency range, for example +0.1 Hz, around the peak in the spectrum.

A signal comprising a lot of information (810) exhibits well marked peaks or significant energy when integrated over a peak. A signal with little information (820) has no peak, and an average signal (815) corresponds to an intermediate result.

In this case, the signal (810) emitted by the elemental gauge that conveys the most information relating to BCG is selected, i.e. the signal emitted by elemental gauge 10 (810) according to this embodiment.

Over successive analyzed time ranges of 20 seconds, more generally between 3 seconds and 60 seconds, the selected elemental gauge is likely to change.

The BCG analysis requires calculating the distance between the signal peaks that correspond to blood expulsion from the heart ventricles. Although these events are graphically visible, and despite prior signal processing as described above, automatic processing remains subject to parasitic phenomena given the conditions of signal acquisition and the environment.

According to a step (460) of exploration, each section corresponding to a time range of the analyzed signal, i.e. comprised between 3 seconds (2T_max) and 60 seconds, preferably of a duration of 20 seconds, is analyzed, according to a sliding window. This exploration aims to detect in such a window the existence of 2 patterns corresponding to heartbeats and the distance separating these patterns.

To this end, a signal exploration variable is defined by times T_min and T_max. As an example, T_min is taken equal to 0.6 s (i.e. 100 bpm) and T_max is taken equal to 1.5 s (i.e. 40 bpm).

A sliding window of width 2T_max is analyzed around each signal exploration point v.

In principle, the treatment aims to:
determining if there are at least two peaks corresponding to heartbeats timely separated by at least T_min and at most T_max in the window;
determining the time distance between these peaks.

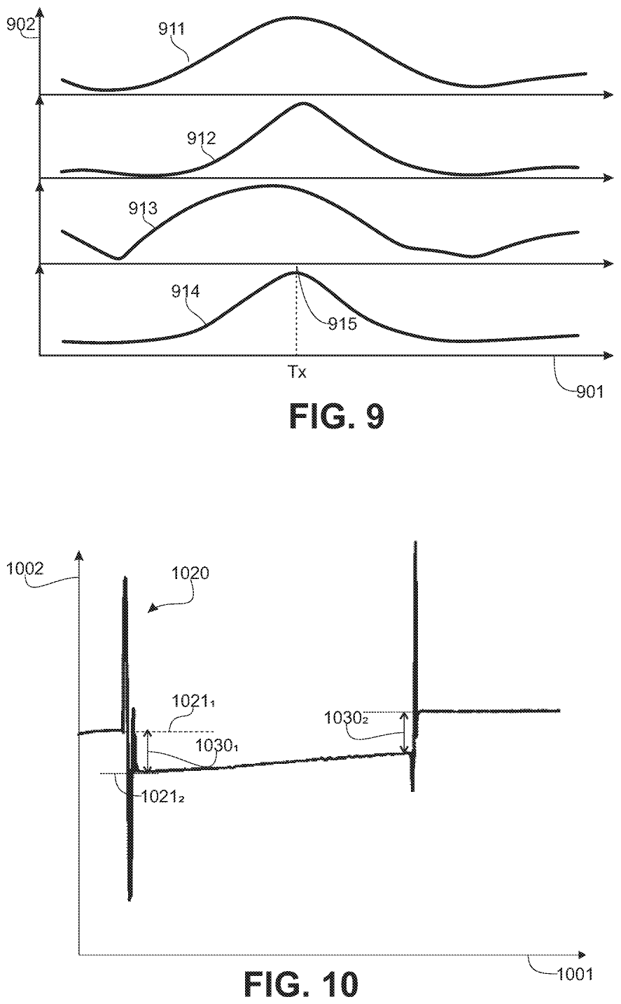

FIG. 9, according to an exemplary implementation, the processing consists in calculating on the window 2T_max, 3 functions having the signal as input:
an autocorrelation function (911);
a modified mean magnitude difference function (AMDF-912)
a maximum amplitude pair function (MAP-913)

Each of these functions defines a probability (902) of two peaks in the signal according to their time distance (901).

By multiplying the probabilities (914) and taking the maximum probability (915) of the distance Tx separating two peaks.

During a verification step (470), the signal window analyzed at point v during the previous step (460) is analyzed between v−Tx and v+Tx so as to locate the maximum amplitude peak in this range. The peak Px is identified for example by its time abscissa in the analyzed time range, preferably of a duration of the order of 20 seconds, and this information is recorded in a timestamped file (479).

If there is already a peak with this same identification in the file, then it means that the pattern has already been identified during the analysis of a previous window. In this case, the determined Tx value is saved in a file. If a new Px peak is detected, then this is a new pattern. In this case, a value Tx corresponding to the median of the values Tx corresponding to the previous exploration windows is associated with the previous peak Px, and this median value of Tx is recorded in the timestamped file (479).

If, however, such a peak is not detected, the Tx value determined is recorded in the timestamped file (479).

The exploration window is then shifted by a value less than T_max and the analysis is restarted.

When the entire measurement range has been processed, the analysis of the file (479) comprising the Tx values makes it possible to determine the heart rate and its variation over that time range, typically 20 seconds.

These can be compared, for example, to acceptable values depending on the age of the individual using the device of the invention and may lead to the generation of alerts based on these results.

According to exemplary embodiments, the alerts are addressed to the individual himself via visual or audio signals.

According to an alternative or complementary embodiment, the alerts

include specific recommendations via a means connected to the device of the invention by a computer link, on the basis of specific patterns identified both on the analysis of breathing and on the cardio analysis or even on the analysis of movements.

Alternatively, or additionally, these alerts are transmitted to a remote monitoring center, for example via an internet or telephone link.

By analyzing the signals, the device and the method of the invention make it possible to detect the presence of an individual on the receiving surface of the device of the invention, to discriminate this presence from that of a dead weight (such as a bag placed on an chair) and to detect specific risky situations depending on the application, such as falling asleep, fainting, or a decrease in alertness, a cardiovascular accident, respiratory distress or intense stress, without this list being exhaustive.

Thus, for certain applications such as the operation of transportation or work equipment, the device advantageously replaces or supplements so-called “dead man” or “automatic monitoring” systems aimed, for example, at stopping the equipment in the event of an operator stroke.

Returning to FIG. 1, according to an embodiment, the device of the invention comprises 2 or more receiving surfaces (111, 112) each equipped with a pressure sensor (121, 122).

According to such a configuration, each receiving surface is used to assess a parameter in a preferred way. For example, the receiving surface located at on the backrest is used to assess respiratory rate while the receiving surface located on the seat is preferentially used for BCG acquisition and analysis.

According to another variant of a device comprising several receiving surfaces simultaneously in contact with the individual, each of these surfaces acts on a specific sensor. The time lag of BCG events detected on distant receiving surfaces makes it possible to assess the speed of blood circulation and consequently a blood pressure of the individual.

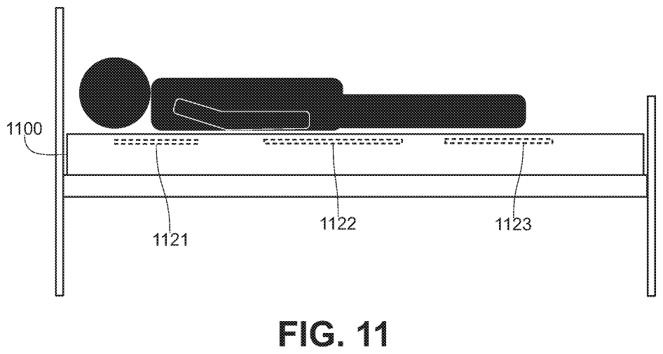

FIG. 11, according to another exemplary embodiment, the device of the invention is integrated into a mattress (1100). One or more sensors (1121, 1122, 1123) are integrated into the mattress and enable measurements of physiological parameters as described above.

More particularly, posture detection makes it possible to highlight points of contact of the individual with the mattress and to prevent bedsores.

The detection of BCG-associated phenomena on two distant sensors also makes it possible to assess a blood circulation speed and consequently a blood tension.

The above description and preferred embodiments show that the device and the method of the invention make it possible to determine measurable parameters relating to the physiological state of an individual when the latter comes into temporary contact with a surface of a piece of furniture (chair, bed, etc.) equipped with the device of the invention, without it being necessary to install specific sensors coming into direct contact with the skin of the individual.

1. A device for assessing the physiological parameters of an individual in temporary contact with the device, which device comprises:
a pressure sensor comprising a deformable test body and a plurality of elemental gauges of high sensitivity linked to the test body and responsive to the deformation of the test body;
a receiving surface capable of coming into contact with the individual and transmitting a force resulting from this contact to the pressure sensor;
computer means, capable of processing a signal coming from the pressure sensor and a computer program for determining at least one item of information among:
a respiratory rate of the individual and an evolution of this respiratory rate;
a heart rate of the individual and an evolution of this heart rate; and
a posture of the individual and an evolution of this posture;
and generating an alert according to the information.

2. The device of claim 1, wherein a gauge of the plurality of elemental gauges comprises an assembly of conductive nanoparticles in an insulating ligand, grafted on a substrate which substrate is bonded to the test body.

3. The device of claim 2, wherein the test body comprises a polycarbonate plate (230) having a thickness less than or equal to 0.5 mm comprising strips (232) defining cutouts (231) and positioned so as to intersect at intersections, the elemental gauges (220) being positioned at the intersections of these strips (232).

4. The device of claim 3, wherein the pressure sensor comprises 12 elemental gauges.

5. The device of claim 1, wherein the device takes the form of a chair (100) comprising a backrest and a seat, and wherein the pressure sensor comprises a backrest sensor (121) inserted into the backrest of the chair, and the receiving surface comprises a backrest receiving surface (111).

6. The device of claim 5, in which the chair backrest (100) comprises a padding.

7. The device of claim 4, wherein the pressure sensor comprises a seat sensor (122) inserted into the seat of the chair, and the receiving surface comprises a seat receiving surface (112).

8. The device of claim 7, wherein the seat comprises a padding.

9. The device of claim 1, wherein the device takes the form of a mattress (1100) comprising a sleeping surface, the pressure sensor (1121, 1122, 1123) comprises a sensor inserted into the mattress and the receiving surface is the sleeping surface.

10. A method for measuring at least one physiological parameter of an individual, implementing the device of claim 1, comprising a step of acquiring and digitizing (410) a signal (520) issued from the plurality of elemental gauges and comprising a continuous part and a pseudo-periodic part, the method comprising over a processing time window, at least one of the steps of:
measuring the pressure (417) exerted by the individual on the gauges of the plurality from the continuous part of the signal after its adjustment (415) for its time drift on the processing time window and deducing therefrom the posture of the individual;
during a pre-processing step (420), extracting a pseudo-periodic part of the signal to remove an influence of movements of the individual, carrying out a smoothing (431) of the pseudo-periodic signal and evaluating a respiratory frequency of the individual by a time distance between two peaks of the smoothed signal;
filtering (432) by a bandpass filter with cut-off frequencies of 0.5 Hz and 20 Hz the pseudo-periodic part of the signal obtained by the pre-processing step (420), selecting the signal thus filtered coming from the gauges of the plurality having a better signal-to-noise ratio, exploring (460) the selected signal by a sliding window and detecting in such a window the existence of 2 patterns corresponding to heart beats and the time distance between these patterns for assessing a heart rate from these data.
